(19) United States
(12) Patent Application Publication
(10) Pub. No.: US 2024/0293655 A1
(43) Pub. Date: Sep. 5, 2024

(54) TOOLS AND METHODS FOR DIRECTLY ACCESSING THE CAROTID ARTERY

(71) Applicant: Contego Medical, Inc., Raleigh, NC (US)

(72) Inventors: Michael Hong, Oakland, CA (US); Steven Tyler, San Jose, CA (US); Ravish Sachar, Raleigh, NC (US)

(21) Appl. No.: 18/592,991

(22) Filed: Mar. 1, 2024

Related U.S. Application Data
(60) Provisional application No. 63/449,196, filed on Mar. 1, 2023.
(60) Provisional application No. 63/523,747, filed on Jun. 28, 2023.

(51) Int. Cl. A61M 39/02; A61F 2/01; A61F 2/966; A61M 25/00; A61M 25/01; A61M 25/02; A61M 39/06

(52) U.S. Cl. CPC A61M 39/0247; A61F 2/0108; A61F 2/011; A61F 2/013; A61F 2/966; A61M 25/0097; A61M 25/0108; A61M 2025/0286; A61M 2039/0258; A61M 2039/0279; A61M 2039/0626

(57) ABSTRACT

Systems, methods, and kits include a sheath assembly including a stabilizer, a stabilizer foot coupled to the stabilizer and including a stabilizer eyelet configured to secure a suture, a gripping mechanism coupled to the stabilizer remote from the stabilizer foot and including a valve eyelet configured to secure the suture, an introducer sheath extending through the gripping mechanism, the stabilizer, and the stabilizer foot, and an entry luer coupled to the introducer sheath. A hemostatic valve includes a hemostatic valve entry port coupled to the entry luer, a bypass port, and an access port. A stopcock includes a stopcock entry port, a collection port, and a stopcock valve providing selective communication between the stopcock entry port and the collection port. A collection bag is connected to the collection port. An angioplasty device can be disposed within the introducer sheath.

### CROSS REFERENCE TO RELATED APPLICATIONS

This application claims the benefit of U.S. Provisional Patent Application No. 63/449,196, filed on Mar. 1, 2023, and U.S. Provisional Patent Application No. 63/523,747, filed on Jun. 28, 2023, the entire contents of which are incorporated herein by reference.

### FIELD

This disclosure relates to carotid artery revascularization, and more particularly to direct carotid artery revascularization via an incision above a patient's clavicle.

### BACKGROUND

Angioplasty catheters and stents are used in catheter-based procedures to open up a blocked vessel and restore blood flow. When treating vascular lesions within the carotid arteries, there is a known risk of embolic material being liberated from the site of treatment during stent deployment or post-deployment balloon dilation of the stent. These embolic particles increase the risk of stroke. To address this risk, many types of vascular embolic filters have been designed. These filters are positioned within the artery past the lesion to be treated and remain in place during the entire procedure. Reverse flow during procedures has been used to reduce the flow of embolic material toward the brain. Reverse flow systems may reintroduce blood to a femoral artery and require aspiration or pumping as well as additional filtration. Femoral reintroduction of blood can increase procedure time and complexity.

### SUMMARY

One embodiment relates to an introducer and flow redirection system for use in carotid angioplasty that includes a sheath assembly including a stabilizer and an introducer sheath, and a flow redirection system in fluid communication with the introducer sheath to collect reverse flow outside a patient's body.

One embodiment relates to an introducer and flow redirection system that includes a sheath assembly including a stabilizer, a stabilizer foot coupled to the stabilizer and including a stabilizer eyelet configured to secure a suture, a gripping mechanism coupled to the stabilizer remote from the stabilizer foot and including a valve eyelet configured to secure the suture, an introducer sheath extending through the gripping mechanism, the stabilizer, and the stabilizer foot, and an entry luer coupled to the introducer sheath. The introducer and flow redirection system further includes a hemostatic valve including a hemostatic valve entry port coupled to the entry luer, a bypass port, and an access port, a stopcock including a stopcock entry port, a collection port, and a stopcock valve providing selective communication between the stopcock entry port and the collection port. A collection bag is connected to the collection port. An angioplasty device can be disposed within the introducer sheath.

One embodiment relates to method of carotid angioplasty including inserting a introducer sheath through a stabilizer including a stabilizer foot and a gripping mechanism, inserting the introducer sheath into a first incision made in the skin above a patient's clavicle and a second incision made in the patient's carotid artery, positioning the stabilizer foot adjacent the first incision and second incision, engaging the skin adjacent the first incision with a suture, securing the suture to an eyelet of the stabilizer foot, and providing a reverse flow from the introducer sheath to a collection bag via a hemostatic valve and a stopcock.

One embodiment relates to a carotid angioplasty kit including a stabilizer including a stabilizer foot, a suture eyelet configured to secure a suture and maintain the stabilizer foot in position relative to an incision, and a gripping mechanism, a introducer sheath sized to be engaged by the gripping mechanism and be received through the stabilizer, a dilator sized to be received in the introducer sheath, a hemostatic valve configured to be coupled to the introducer sheath, a collection bag in fluid communication with the hemostatic valve, and an angioplasty device sized to be disposed within the introducer sheath.

### DESCRIPTION OF DRAWINGS

The device is explained in even greater detail in the following drawings. The drawings are merely exemplary and certain features may be used singularly or in combination with other features. The drawings are not necessarily drawn to scale.

FIG. 1A is a side view of sheath assembly system according to some embodiments.

FIG. 1B is a side view of a dilator of the sheath assembly system of FIG. 1 according to some embodiments.

FIG. 2A is a side view of a rotating hemostatic valve (RHV) of the sheath assembly of FIG. 1 according to some embodiments.

FIG. 2B is a side view of an alternative, passive hemostatic valve that can replace the RHV of FIG. 2A in the sheath assembly of FIG. 1 according to some embodiments.

FIG. 3 is a side view of a three-way stopcock of the sheath assembly of FIG. 1 according to some embodiments.

FIG. 4 is a front view of an introducer and flow redirection system including the sheath assembly system of FIG. 1, according to some embodiments.

FIG. 5 is a side view of an angioplasty device in an insertion arrangement according to some embodiments.

FIG. 6 is a side view of a handle of the angioplasty device of FIG. 5 according to some embodiments.

FIG. 7 is a side view of the angioplasty device of FIG. 5 in a filter positioning arrangement according to some embodiments.

FIG. 8 is a side view of the angioplasty device of FIG. 5 in a filter deployed arrangement according to some embodiments.

FIG. 9 is a side view of the angioplasty device of FIG. 5 in a stent positioning arrangement according to some embodiments.

FIG. 10 is a side view of the angioplasty device of FIG. 5 in a stent deployed arrangement according to some embodiments.

FIG. 11 is a side view of the angioplasty device of FIG. 5 in a balloon expanded arrangement according to some embodiments.

FIG. 12 is a side view of the angioplasty device of FIG. 5 in a balloon retracted arrangement according to some embodiments.

FIG. 13 is a side view of the angioplasty device of FIG. 5 in a filter retracted arrangement according to some embodiments.

FIG. 14 is a perspective view of a flow redirection system according to some embodiments.

FIG. 15 is a schematic representation of the angioplasty device of FIG. 5 and the flow control of FIG. 14 installed via an introducer sheath through an incision above a patient's clavicle according to some embodiments.

FIG. 16 is a schematic representation of the angioplasty device of FIG. 15 in the insertion arrangement according to some embodiments.

FIG. 17 is a schematic representation of the angioplasty device of FIG. 15 in the insertion arrangement and a clamp installed on the patient's carotid artery to provide reverse flow toward the collection system according to some embodiments.

FIG. 18 is a schematic representation of the angioplasty device of FIG. 15 in the insertion arrangement and in position to place a stent according to some embodiments.

FIG. 19 is a schematic representation of the angioplasty device of FIG. 15 retracted and the stent placed in the carotid artery according to some embodiments.

### DETAILED DESCRIPTION

The following description of certain examples of the inventive concepts should not be used to limit the scope of the claims. Other examples, features, aspects, configurations, embodiments, and advantages will become apparent to those skilled in the art from the following description. As will be realized, the device and/or methods are capable of other aspects, all without departing from the spirit of the inventive concepts. Accordingly, the drawings and descriptions should be regarded as illustrative in nature and not restrictive.

Systems, methods, and kits described herein provide direct carotid artery revascularization with selective flow reversal involving single crossing of a lesion with a three-in-one (stent/balloon/filter) angioplasty device. A non-exhaustive list of advantages includes a swift workflow, no arch navigation, flow reversal, local anesthesia, and a low profile device. The arrangement of devices described herein allow for collection outside a patient's body during reverse flow via gravity and without requiring aspiration. The result is a procedure that is faster, safer, and cheaper than conventional.

Embodiments of introducer and flow redirection system for use in carotid angioplasty procedures generally include a stabilizer including a stabilizer foot, a suture eyelet configured to secure a suture and maintain the stabilizer foot in position relative to an incision, and a gripping mechanism. An introducer sheath is sized to be engaged by the gripping mechanism and be received through the stabilizer. A dilator is sized to be received in the introducer sheath. A hemostatic valve is coupled to the introducer sheath and a collection bag is in fluid communication with the hemostatic valve. An angioplasty device is sized to be disposed within the tool sheath. Under local anesthesia and conscious sedation, the introducer and flow redirection system is used to assist introduction of an angioplasty device directly into the common carotid and with reverse blood flow control during the angioplasty procedure. The common carotid is initially accessed with a micro-puncture wire (advanced into the external carotid) and dilator under ultrasound guidance. An introducer sheath with a rotating hemostatic valve at its proximal end is then inserted partially into the vessel over the wire and fixed to the skin. The dilator is removed, and the angioplasty device is then inserted and the stent placed. During placement of the stent, flow is reversed and captured in the collection bag via gravity drain. No additional aspiration, pumping, or suction devices are required. The collection bag is then disposed of and no reverse flowed fluids are returned to the patient.

To prevent unnecessary radiation exposure, imaging can be limited to ultrasound during the insertion of the introducer. Where fluoroscopy is used, it need not be initiated until the introducer sheath is in place below the bifurcation of the carotid artery. Once fluoroscopic imaging begins, the fluoroscopy beam can be focused on the treatment site at the bifurcation to avoid unnecessary radiation at the insertion site.

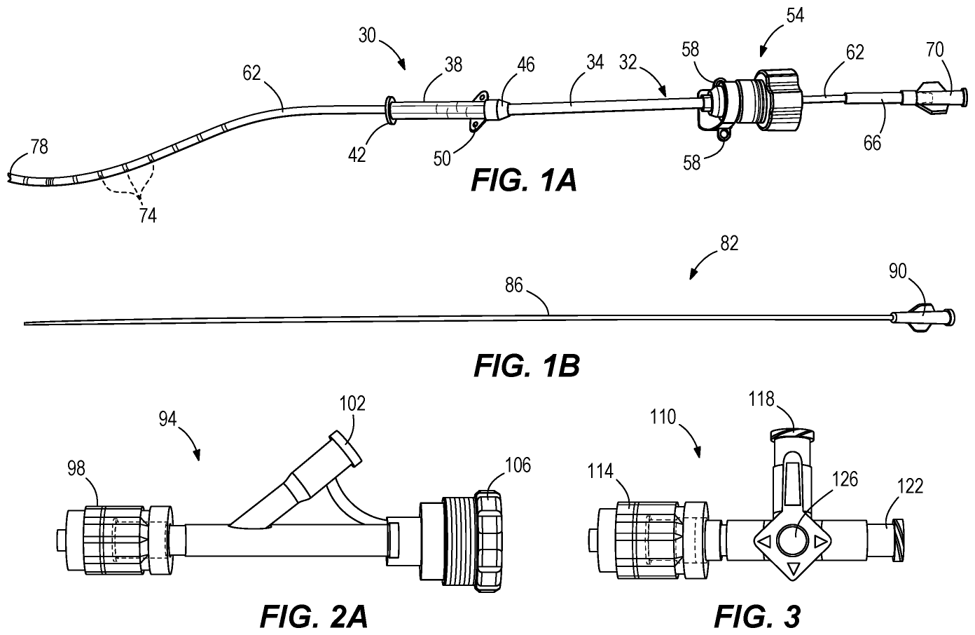

As shown in FIG. 1, a sheath assembly 30 includes a stabilizer 32 that defines a size between about five French (5 Fr) and about nine French (9 Fr) in inner diameter and can be sized and configured for use as part of a direct access kit. In some embodiments, the stabilizer 32 includes a rigid or semi-rigid shaft 34 and a lubricious inner liner. An elastomeric outer layer covers the shaft 34, in some embodiments. For example, the shaft 34 can include a central metal layer (such as, but not limited to, stainless steel), or the shaft 34 can include a central metal coil, if heightened flexibility is desired. In some embodiments, the coil is replaced with a braid or a laser cut stainless steel hypotube. The lubricious inner liner of shaft 34 can include, for example, a polytetrafluoroethylene material. The elastomeric outer layer of shaft 34 can include, for example, a PEBAX® material. In some embodiments, the stabilizer 32 defines an internal diameter of about 11 French (11 FR) and is structured for use with the Neuroguard IEP Direct Access System.

A stabilizer foot 38 is supported by the stabilizer 32 and includes a distal end 42 that interacts directly with a patient's body (and in some embodiments, comes into direct contact with the patient's carotid artery), a stabilizer collar 46 that maintains the stabilizer foot 38 in position relative to the stabilizer 32, and two stabilizer eyelets 50 that are shaped to receive and secure a suture. In some embodiments, the stabilizer foot 38 includes a silicone material. In some embodiments, the stabilizer foot 38 is formed from a medical grade injection molded silicone. In some embodiments, more than two or less than two stabilizer eyelets 50 are provided. In some embodiments, the stabilizer eyelets 50 include speed hooks, protrusions, detent structures, or another feature to provide convenient and secure engagement with the suture. In some embodiments, the stabilizer collar 46 is sized for a weak interference fit with the shaft 34 so that the user can grasp and move the stabilizer foot 38 along the shaft 34 with force, and the stabilizer foot 38 will generally be maintained in position along the shaft 34 when not being forced to move by the user.

A gripping mechanism 54 is engaged with an end of the stabilizer 32 opposite the stabilizer foot 38. In the embodiment shown in FIG. 1, the gripping mechanism takes the form of a Tuohy Borst valve. The gripping mechanism 54 can be actuated between a locked position and an unlocked position and provides a sealed entry to the stabilizer 32. The gripping mechanism 54 also includes two valve eyelets 58 shaped to receive and secure a suture. In some embodiments, the two valve eyelets 58 are aligned with the two stabilizer eyelets 50 and cooperate therewith to secure and engage the suture. In some embodiments, more than two or less than two valve eyelets 58 are provided. In some embodiments, the valve eyelets 58 include speed hooks, protrusions, detent structures, or another feature to provide convenient and secure engagement with the suture.

Introducer sheath 62 can include three layers of material (e.g., Pebax® outer layer, Nitinol (NiTi) coil, and PTFE liner) which provides a balance of flexibility and column strength and allows internal passage of devices while providing adequate clearance for the flow of blood away from the patient during procedures. In some embodiments, the coil is replaced with a stainless steel coil, a braid, or a laser cut stainless steel hypotube. In some embodiments, the introducer sheath 62 defines an outer diameter of about ten French (10 FR) and is sized to be sealingly engaged by the gripping mechanism 54 so that the relative position of the introducer sheath 62 can be locked to the stabilizer 32. The introducer sheath 62 includes a strain relief 66 and an entry luer 70.

In some embodiments, the introducer sheath extends a length of from about 30 centimeters (30 cm) to about 45 centimeters (45 cm) as measured from a proximal end to a distal end. In some embodiments, the introducer sheath extends a length of from about 35 cm to about 40 cm. The lumen of the introducer sheath 62 can extend continuously between the distal end and the proximal end of introducer sheath 62, such that blood flowing into the distal end is carried through the entire length of introducer sheath 62.

Printed indicators 74 along a distal segment of the introducer sheath 62 allow visualization of the placement of the introducer sheath 62 into the common carotid artery. A radiopaque marker 78 on a distal end of the introducer sheath 62 indicates the location of a sheath tip in the patient's body. In some embodiments, the sheath tip (distal end) can be curved to facilitate insertion into the artery at an angle as the tip is intended to bend away from the arterial wall. Advantageously, the distance the sheath is inserted into the artery can vary (and/or can be adjusted) between about one centimeter (1 cm) and about five centimeters (5 cm).

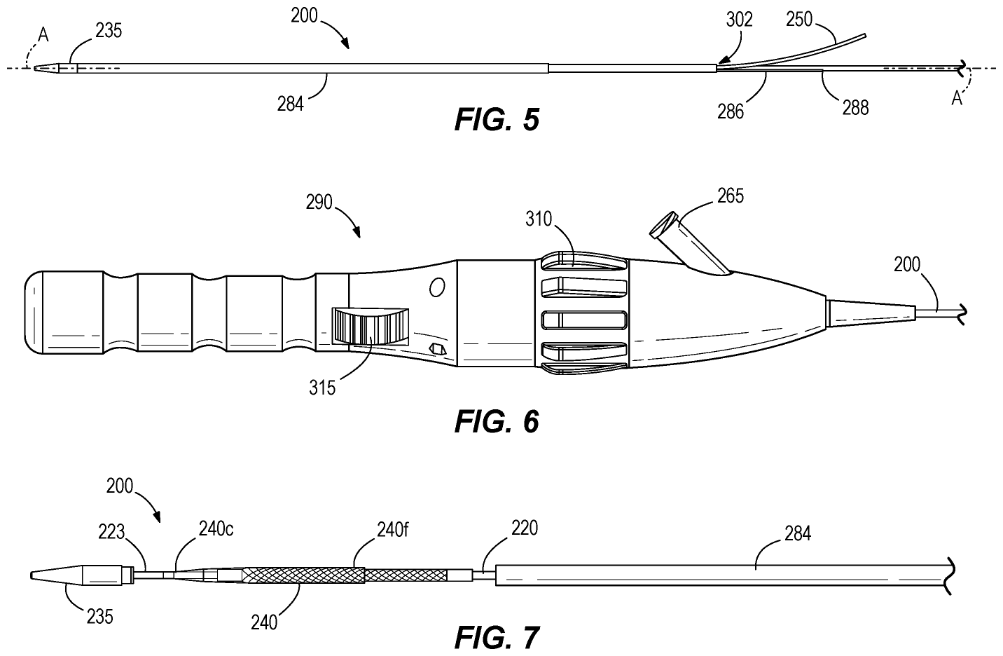

As shown in FIG. 1A, a dilator 82 is designed to go over a 0.035″ to 0.038″ guidewire 250 (see FIG. 5). The dilator 82 includes a dilator shaft 86 with a tapered distal end for smooth transition between the wire and the dilator 82, and the dilator 82 and the introducer sheath 62. The dilator 82 also includes a dilator hub 90.

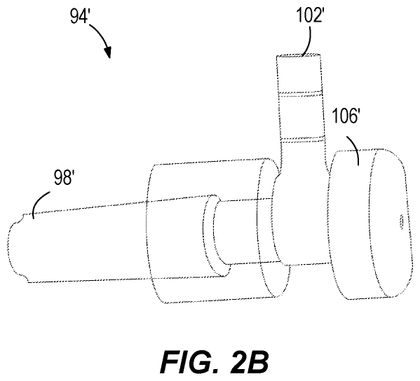

As shown in FIG. 2A, a rotating hemostatic valve (RHV) 94 is structured to be attached to the entry luer 70 at a proximal end of the introducer sheath 62. The RHV 94 includes an RHV entry port 98, an RHV bypass port 102, and an RHV access port 106. The RHV 94 allows for introduction of devices through the introducer sheath 62 while providing hemostasis while the RHV bypass port 102 connects to a flow redirection system as will be described in detail below.

An alternate embodiment utilizes a hemostatic valve with a passive valve that relies on a deformable material to create a fluid seal around an inserted device. For example, as shown in FIG. 2B, a passive hemostatic valve 94′ is structured to be attached to the entry luer 70 at a proximal end of the introducer sheath 62. The passive hemostatic valve 94′ includes an entry port 98′, a bypass port 102′, and an access port 106′. The passive hemostatic valve 94′ allows for introduction of devices through the introducer sheath 62 while providing hemostasis while the bypass port 102′ connects to a flow redirection system as will be described in detail below.

As shown in FIG. 3, a stopcock 110 is structured to attach to the RHV bypass port 102 and provide selective reverse flow. The stopcock 110 includes a stopcock entry port 114, a stopcock auxiliary port 118, a stopcock collection port 122 and a stopcock valve 126 that is moveable between a stopcock off position where flow is inhibited between the stopcock entry port 114 and the stopcock collection port 122 and a stopcock on position where flow is allowed between the stopcock entry port 114 and the stopcock collection port 122. In some embodiments, the stopcock on position includes a low flow position and a high flow position that provide relatively different flow rates through the stopcock 110.

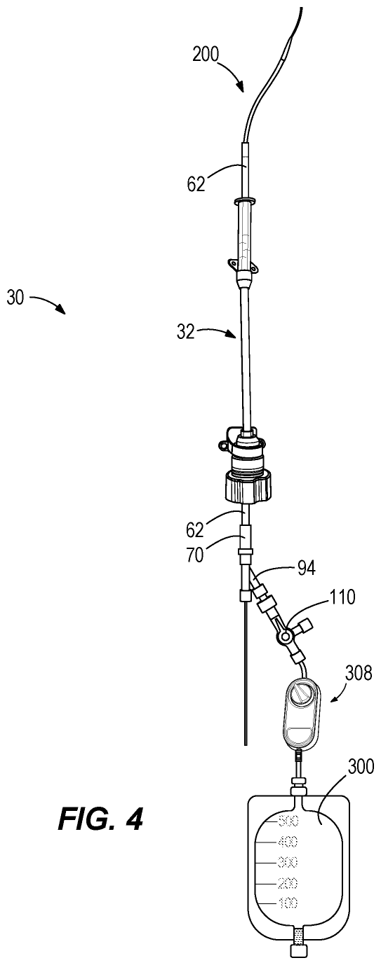

As shown in FIG. 4, an introducer and flow redirection system includes the sheath assembly 30, the RHV 94, the stopcock 110, an angioplasty device 200, and a flow redirection system 300.

As shown in FIG. 5, the angioplasty device 200 is usable with the introducer sheath 62. According to the implementation shown in FIGS. 5-13, the angioplasty device 200 includes a catheter 220 having one or more axial lumens extending at least partially through the catheter, an integrated filter assembly 240, an expandable balloon 260, a stent 280, and an axially movable sheath 284. FIG. 5 is a side view of the angioplasty device 200 with the sheath 284 covering the filter assembly 240, stent 280, and balloon 260. Examples of angioplasty devices with integrated embolic protection are shown and described in U.S. Pat. Nos. 9,968,472 and 10,849,730 and can be utilized with the introducer sheath assembly embodiments and flow redirection system embodiments disclosed herein. U.S. Pat. No. 9,968,472 and U.S. Pat. No. 10,849,730 are hereby incorporated by reference in their entireties.

The angioplasty device 200 includes a distal tip 235 that is conical or frusto-conically shaped to facilitate penetration through the body. The tip 235 defines a guidewire port through which a guidewire 250 extends along axis A during placement of the angioplasty device 200 within the body. The distal tip 235 according to one implementation includes a low durometer material, such as Pebax®. However, in other implementations, the distal tip 235 includes other suitable shapes (e.g., spherical or hemispherical, pyramidal, blunted) depending on the intended path of the distal tip 235 through the body. The angioplasty device 200 also includes a sheath wire 286 accessed via a sheath wire port 288, and a guide wire port 302.

As shown in FIG. 6, a handle 290 is coupled to a proximal end of the catheter 220 and includes controls (e.g., buttons, knobs, etc.) that are coupled to one or more of the filter assembly 240, the sheath wire 286, and/or the guidewire 250 to allow the user to actuate the filter 240, the sheath 284, and/or the guide wire 250. The handle 290 includes knobs 310, 315 for controlling the filter assembly 240 and the sheath wire 286, respectively. Actuation of the knobs 310, 315 in one direction causes the respective wires to be tensioned proximally, and actuation of the knobs 310, 315 in the opposite direction releases tension on the wires. In addition, the handle 290 defines a proximal balloon inflation port 265 that is in fluid communication with balloon inflation lumens and the balloon 260 to provide air/fluid to the balloon 260 for expansion.

As shown in FIGS. 7-13, the angioplasty device 200 including the filter assembly 240 and stent 280 are deployed and the filter assembly 240 is then collapsed and removed with the angioplasty device 200, leaving the stent 280 in place within the body.

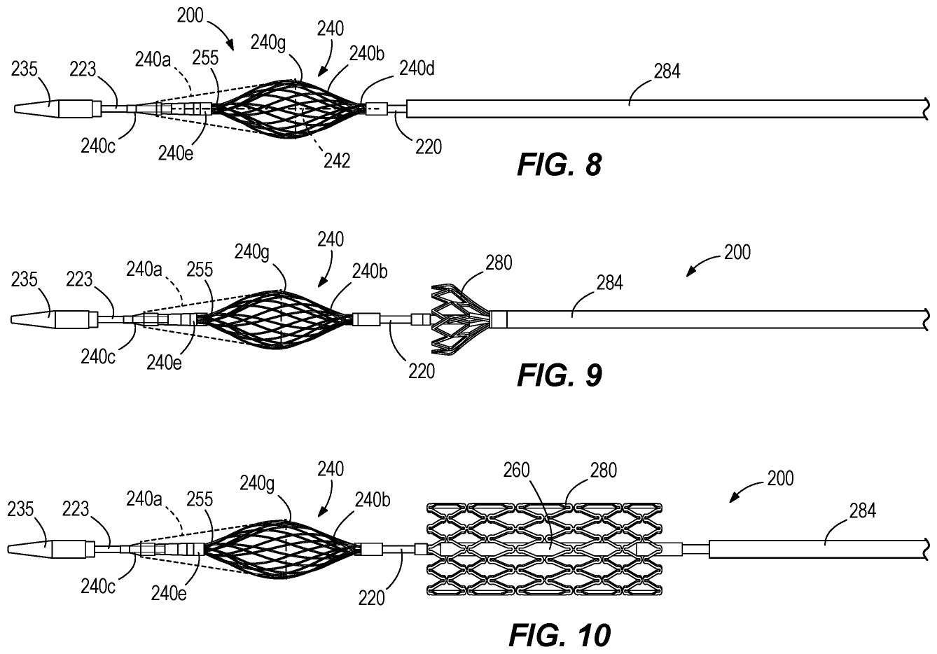

As shown in FIG. 7, the filter assembly 240 is coupled to the catheter 220 adjacent a distal end 223 of the catheter 220 and is disposed axially proximal to the tip 235. The filter assembly 240 is moveable between an expanded configuration (see FIG. 8) and an unexpanded configuration shown in FIG. 7. The filter assembly 240 in the unexpanded configuration, which is illustrated in FIGS. 7 and 13, is sized and configured for insertion and passage through a blood vessel. In the expanded configuration, illustrated in FIGS. 8-12, the filter assembly 240 is sized and configured to capture emboli within the bloodstream. For example, at least a portion of the filter assembly 240 in the expanded configuration extends across a diameter of the blood vessel to catch emboli that may be flowing through the bloodstream.

As shown in FIG. 8, the filter assembly 240 includes a filter membrane 240a and a filter frame 240b. The filter membrane 240a is frusto-conically shaped, and the filter frame 240b is egg shaped. A conical tip 240c of the membrane 240a is fixedly coupled around a distal portion of the catheter 220, and a distal end 240e of the filter frame 240b is disposed proximally of the conical tip 240c of the membrane 240a and is slidably coupled around the distal portion of the catheter 220. A proximal portion 240f of the filter membrane 240a is fixedly coupled to a central portion 240g of the filter frame 240b, such as via thermal or chemical bonding or another suitable coupling mechanism. And, a proximal portion 240d of the filter frame 240b is fixedly coupled around the distal portion 220b of catheter 220. In other implementations, the shape of the membrane and/or filter frame may be different and may be based at least in part on the anatomy in which the filter assembly is to be disposed.

A filter activation wire 242 extends through the filter activation wire lumens and a distal end of the filter activation wire 242 is coupled to the distal end 240e of the filter frame 240b. Tensioning the filter activation wire 242 in the proximal direction causes the distal end 240e of the filter frame 240b to move proximally, which causes the filter assembly 240 to move from the unexpanded configuration to the expanded configuration. Similarly, releasing tension on the filter activation wire 242 allows the filter assembly 240 to move into the unexpanded configuration. The filter assembly 240 is self-collapsing. In other words, the filter assembly 240 is biased toward the unexpanded configuration and is forced toward the expanded configuration against the bias. In the expanded position, an outer diameter of the filter frame 240b around the central portion 240g and an outer diameter of the proximal portion 240f of the filter membrane 240a correspond to an inner diameter of an artery or vessel to ensure that any embolic material is captured by the filter assembly 240. In addition, the filter membrane 240a and the filter frame 240b allow blood/fluid to flow therethrough.

In some embodiments, the filter membrane 240a comprises a biocompatible, elastic polymer sheet (e.g., polyurethane) that defines an array of pores. In some embodiments, the pores are between forty microns and one hundred microns in diameter, which allows blood to flow through but captures small particulates. In some embodiments, the pores are between forty microns and sixty microns. In some embodiments, the pores are between about forty microns and about fifty microns. In some embodiments, the pores are about forty microns. In some embodiments, the pores are formed by laser drilling. In some embodiments, the filter frame 240b comprises a biocompatible, expandable structure that defines a plurality of pores. The pores of the filter frame 240b are larger than the pores defined by the filter membrane 240a. The filter frame 240b, according to some implementations, includes a material having shape memory properties, such as a braided nitinol structure or a laser cut nitinol tube structure. Other suitable biocompatible materials include titanium and titanium alloys, stainless steel, platinum, gold, or other metals, as well as ceramics or polymers. In some embodiments, the filter frame 240b has a memory of the unexpanded configuration such that when tension on the filter activation wire 242 is released, the filter frame 240 returns toward its unexpanded configuration, capturing any embolic materials that have been captured within the filter assembly 240.

The expandable balloon 260 is disposed adjacent the proximal end 240d of the filter frame 240b and air and/or fluid is provided to the balloon 260 for inflation via the balloon inflation lumens. The stent 280 is disposed over at least a portion of the balloon 260. In some embodiments, the stent 280 is a self-expanding stent constrained in place over at least a portion of the balloon 260. In some embodiments, the stent 280 is a controlled/directed expansion stent. For example, in such implementations having a controlled/directed expansion stent, a stent deployment wire is coupled to the stent to direct expansion and contraction of the stent. In some embodiments, the stent 280 is a balloon-expandable stent. The selection of the type, dimensions, and/or radial strength of the stent 280 is based at least in part on the anatomy in which the stent 280 is being deployed.

As shown in FIG. 8, the stent 280 is constrained by the movable sheath 284. Exemplary sheaths 284 include a wire, coiled wire, polymer filament, or polymer braid sheath. For example, in some implementations, the sheath 284 includes an inner polymer layer (e.g., PTFE composite) to reduce friction with components disposed radially within the sheath 284, a structural sheath layer (e.g., a wire, coiled wire, polymer filament, or polymer braid sheath layer (e.g., a braided stainless steel sheath layer)) to maintain the radial strength of the sheath 284, and an outer polymer layer (e.g., nylon) to protect the structural sheath layer. In some embodiments, the coil is replaced with a braid or a laser cut stainless steel hypotube. In some embodiments, the sheath 284 is a 6 F sheath/8 F guide compatible sheath. In some embodiments, the sheath 284 does not extend over the filter assembly 240.

As shown in FIG. 8, the sheath 284 is moved axially to expose the filter assembly 240 by pulling the sheath wire proximally to expose the filter assembly 240. Then, the filter assembly 240 is deployed into the expanded configuration by tensioning the filter activation wire 242. Deploying the filter assembly 240 prior to deploying the stent 280 allows the filter assembly 240 to catch any embolic material that is dislodged during deployment of the stent 280.

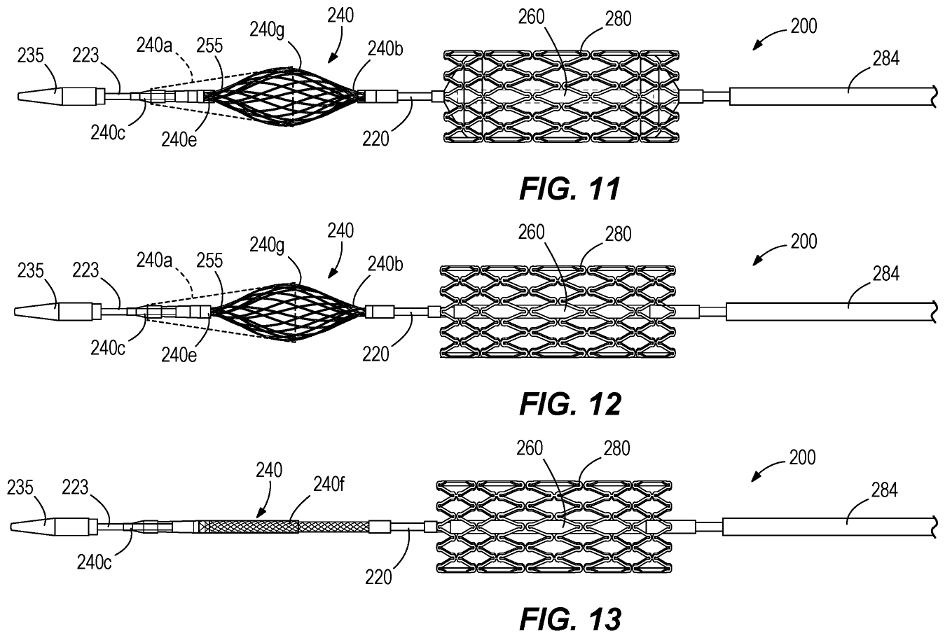

As shown in FIG. 9, the sheath 284 is moved further axially to expose the stent 280. As shown in FIG. 10, with the sheath 284 disposed proximally of the stent 280, the stent 280 expands radially based on the memory properties of the material of the stent 280 for implementations that include a self-expanding stent. As shown in FIG. 11, the balloon 260 is inflated against an inner surface of the stent 280 such that the stent 280 is further radially expanded against the vessel wall (post-dilatation expansion). This step of post-dilatation may be repeated to expand the stenosed region of the artery and expand the stent 280 further radially against/toward the vessel wall. For example, the post-dilatation step may be repeated until the vessel is fully dilated. For example, the balloon 260 is inflated (or deflated) via fluid/air provided to (or removed from) a central chamber of the balloon 260 via port 265 of the handle 290. After the vessel is fully dilated, the balloon 260 is deflated as shown in FIG. 12. As shown in FIG. 13, tension in the filter activation wire 242 is then released, and the filter membrane 240a and the filter frame 240b are collapsed by releasing the filter activation wire 242, which securely capture any embolic material captured by the filter assembly 240. The angioplasty device 200 can then be removed leaving the stent 280 in place. Because the filter assembly 240 is able to capture and hold the embolic material upon release of the filter activation wire 242, it is not necessary to move the sheath 284 distally over the filter assembly 240 prior to removal of the device 200 from the body, which reduces the time required for the procedure.

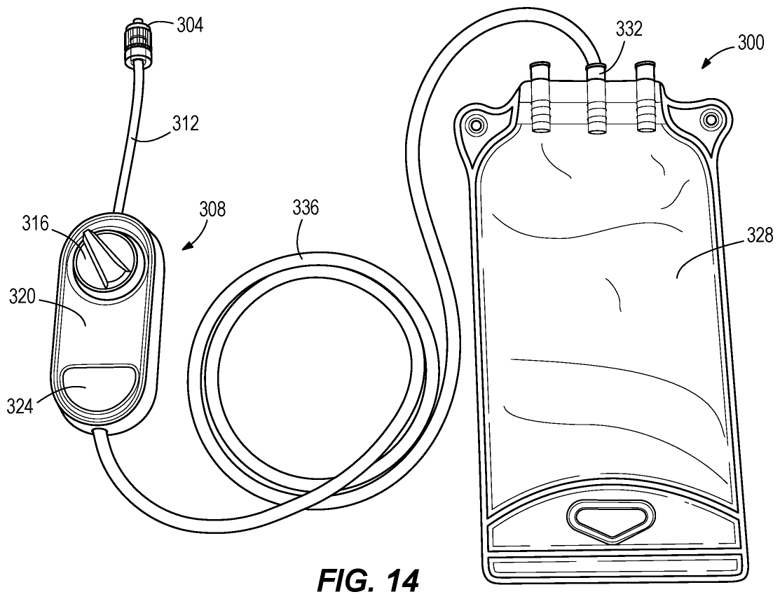

As shown in FIG. 14, the flow redirection system 300 includes a rotating collection port 304 structured to engage the stopcock collection port 122, and a flow control handle 308 connected to the collection port 304 by an entry lumen 312. The flow control handle 308 includes a flow control switch 316, a flow restrictor 320 (e.g., a 0.016″ ID steel tube), and a flow visualization chamber 324. A collection bag 328 receives flow from the flow visualization chamber 324 of the flow control handle 308 via bag plugs 332 and a collection lumen 336.

The flow redirection system 300 modulates blood flow to provide minimal blood loss and consistent flow rate regardless of whether the lumen of the introducer sheath 62 is filled or empty. The flow redirection system 300 further avoids the need for a second surgical puncture to return blood flow to the patient. The flow redirection system 300 provides embolic protection during an angioplasty procedure. When using the flow redirection system 300, embolic debris generated during angioplasty and stenting procedures is directed out of the body by reversing the flow of blood (away from cranial circulation).

In some embodiments, the flow redirection system 300 is intended for use with the Neuroguard IEP® Direct Access System provided by Contego Medical, Inc. and improves embolic protection during the procedure. The flow redirection system 300 allows the initiation of flow reversal by switching the stopcock valve 126 from the off position to the on position. The reversed blood is directed out of the introducer sheath 62, through the RHV 94, through the stopcock 110, and into the flow redirection system 300. The flow control switch 316 modulates the flow rate by allowing the user to toggle between an “OFF,” a “LOW,” and a “HIGH” flow settings. In some embodiments, the flow control switch 316 provides more preset flow rates, or an adjustable flow rate. The flow control switch 316 starts on the off flow setting or on the low flow setting. During the procedure, the carotid artery is clamped with the introducer sheath 62 in place. To maintain flow reversal and embolic protection at a consistent rate, the flow control switch 316 is changes to the “HIGH” flow setting at the time the angioplasty device 200 is introduced. The presence of angioplasty device 200 within the introducer sheath 62 restricts the flow rate of blood and using the “HIGH” flow setting offsets this restriction and maintains constant flow. The reversed blood then flows through the flow restrictor 320 which further modulates the flow rate and into the flow visualization chamber 324 which allows for visualization of continuous blood flow and confirmation of embolic protection. Finally, the blood flows through the collection lumen 336 and accumulates in the collection bag 328. The collected blood is disposed of after the procedure and is not returned to the patient. In some implementations, a single flow path is provided through the flow redirection system 300 and the single flow path can be enlarged or restricted to achieve different flow rates (e.g., via manipulation of the flow control switch 316 between OFF, LOW, and HIGH). In some implementations, the flow restrictor 320 includes a pinch valve that is actuated via the flow control switch 316 to affect flow rate through the flow redirection system 300.

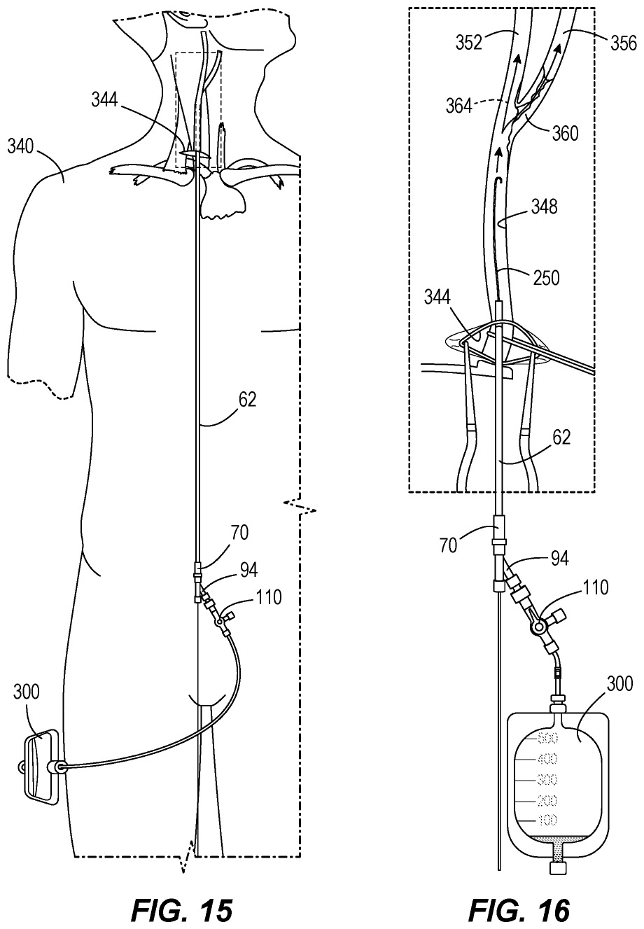

As shown in FIG. 15, The introducer and flow redirection system 30 can be used to install the stent 280 in a patient 340 via a first incision 344 above a clavicle of the patient 340. As shown in FIG. 16, the guidewire 250 can be introduced through a second incision 346 in a common carotid artery 348 and directed to an internal carotid artery 352 or an external carotid artery 356 toward a blockage 360. Direction 364 defines a normal direction of blood flow. A suture 376 is engaged around the common carotid artery 348 and secured to the stabilizer eyelets 50 of the stabilizer foot 38 and the valve eyelets 58 of the gripping mechanism 54 so that the stabilizer 32 is maintained in position relative to the incision 344. In some embodiments, physicians can use either, both, or neither of the stabilizer eyelets 50 and the valve eyelets 58. When using the stabilizer eyelets 50 on the foot, the sutures 376 are placed through the skin just outside of the access site. When using the sutures 376 on the valve eyelets 58 of the gripping mechanism, the sutures 376 are placed either through the skin or the sterile drape. In some embodiments, the valve eyelets 58 on the gripping mechanism are about ten centimeters (10 cm) proximal to the stabilizer eyelets 50 on the foot. The stabilizer foot 38 provides stability to the stabilizer 32 and the other components of the stenting system 30 during the procedure. Once the introducer sheath 62 is positioned, the stabilizer foot 38 is slid into location. A front end (distal) 42 of the stabilizer foot 38 can be angled as it is designed to rest on top of the common carotid artery 348 during the procedure. Once the distal end 42 is in place, the gripping mechanism 54 at the proximal end of the stabilizer 32 locks the introducer sheath 62 in place to prevent movement of the devices during the procedure. Placement of the introducer sheath 62 is aided by the printed indicators 74. Once the introducer sheath 62 is in position, accurate placement can be confirmed with imaging based on the radiopaque marker. Imaging is accomplished remote from the incision 344 and advantageously reduces the impact on the user or other tools positioned adjacent the incision 344. The gripping mechanism 54 helps maintain the introducer sheath 62 in place relative to the stabilizer 32. The stabilizer eyelets 50 and the valve eyelets 58 allow suture placement for securement and keep everything in place.

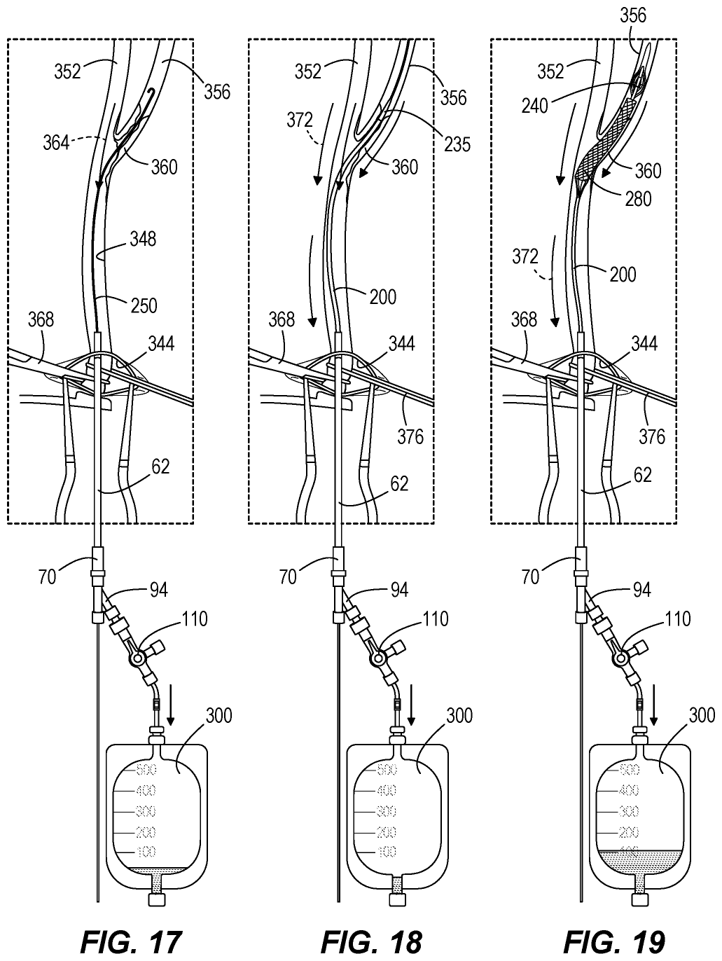

As shown in FIG. 17, with the introducer sheath 62 introduced to the common carotid artery 348, a clamp 368 is applied to the carotid artery 348 to enable reverse flow 372. The lesion is not crossed before flow reversal is applied. Clamping the common carotid artery 348 inhibits forward blood flow on that side (e.g., left or right side of head), causing blood to flow from the other side through the Circle of Willis and into the clamped side, thus reversing flow. Insertion of the sheath 62 into the common carotid artery 348 and exposing it to the ambient environment causes a pressure differential between the higher arterial pressure and the lower atmospheric pressure. In addition, hanging the blood collection bag 328 below the level of the carotid artery 348 allows gravitational force to drain blood into the bag 328. Combined, the effects of clamping reversal, pressure gradient, and gravitational force effectively reverse the direction of blood flow.

The position of the collection bag 328 and/or the flow control switch 316 can be used to control the reverse flow 372 rate. In some embodiments, the collection bag 328 is placed about twenty centimeters (20 cm) to about forty centimeters (40 cm) (including about 20 cm, about 25 cm, about 30 cm, about 35 cm, and about 40 cm) below the common carotid artery 348. In some embodiments, any height differential greater than zero can be used to cause reverse flow. Active aspiration is not necessary to achieve reverse flow with the system and methods disclosed herein.

As shown in FIG. 18, the angioplasty device 200 is then introduced. As shown in FIG. 19, the stent 280 is placed and the angioplasty device 200 is removed. The embolic material jogged loose during placement of the stent 280 is captured and removed from the patient 340 by the filter device 240. Once the angioplasty device 200 is removed, the clamp 368 can be removed and normal flow 364 re-established. In some embodiments, about forty to sixty cubic centimeters (40-60 cc) of blood is collected in the collection bag 328 and is discarded after the procedure.

Generally, filter device 240 acts as a redundant safety mechanism in cases where flow reversal is not fully effective at removing particles. Also, there may be times during a procedure when blood flow reversal may need to be ceased. During those times, open filter device 240 catches any embolic particles present in the forward blood flow. For example, at some points during the procedure, the procedure site may need to be reimaged (to create a new vascular road map). This can happen, for example, when a patient moves. Forward blood flow is necessary to move contrast agent through the vasculature so that new images can be acquired. Reverse flow can be reinstated once the imaging is complete.

In some embodiments, components described herein are provided as a kit of parts for use in a carotid artery angioplasty or stenting procedure. A direct access carotid angioplasty kit includes the stabilizer 32 including the stabilizer foot 38, the suture eyelets 50 and or 58 configured to secure the suture 376 and maintain the distal end 42 of stabilizer foot 38 in position relative to the incision 344, and the gripping mechanism 54. The kit also includes the introducer sheath 62 sized to be engaged by the gripping mechanism 54 and be received through the stabilizer 32, the dilator 82 sized to be received in the introducer sheath 62, the hemostatic valve 94 configured to be coupled to the introducer sheath 62, the collection bag 328 for placement in fluid communication with the hemostatic valve 94, and the angioplasty device 200 sized to be disposed within the introducer sheath 62.

For purposes of this description, certain advantages and novel features of the aspects and configurations of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Although the operations of exemplary aspects of the disclosed method may be described in a particular, sequential order for convenient presentation, it should be understood that disclosed aspects can encompass an order of operations other than the particular, sequential order disclosed. For example, operations described sequentially may in some cases be rearranged or performed concurrently. Further, descriptions and disclosures provided in association with one particular aspect or implementation are not limited to that aspect or implementation, and may be applied to any aspect or implementation disclosed. It will understood that various changes and additional variations may be made and equivalents may be substituted for elements thereof without departing from the scope of the invention or the inventive concept thereof. Certain aspects and features of any given aspect may be translated to other aspects described herein. In addition, many modifications may be made to adapt a particular situation or device to the teachings of the invention without departing from the essential scope thereof. Therefore, it is intended that the invention not be limited to the particular implementations disclosed herein, but that the invention will include all implementations falling within the scope of the appended claims.

Features disclosed in this specification (including any accompanying claims, abstract, and drawings), and/or all of the steps of any method or process so disclosed, may be combined in any combination, except combinations where at least some of such features and/or steps are mutually exclusive. The claimed features extend to any novel one, or any novel combination, of the features disclosed in this specification (including any accompanying claims, abstract, and drawings), or to any novel one, or any novel combination, of the steps of any method or process so disclosed.

As used in the specification and the appended claims, the singular forms “a”, “an”, and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about”, it will be understood that the particular value forms another aspect. It will be further understood that the endpoints of each of the ranges are significant both in relation to the other endpoint, and independently of the other endpoint. The terms “about” and “approximately” are defined as being “close to” as understood by one of ordinary skill in the art. In one non-limiting aspect the terms are defined to be within 10%. In another non-limiting aspect, the terms are defined to be within 5%. In still another non-limiting aspect, the terms are defined to be within 1%.

“Optional” or “optionally” means that the subsequently described event or circumstance may or may not occur, and that the description includes instances where said event or circumstance occurs and instances where it does not.

The terms “coupled”, “connected”, and the like as used herein mean the joining of two members directly or indirectly to one another. Such joining may be stationary (e.g., permanent) or moveable (e.g., removable or releasable). Such joining may be achieved with the two members or the two members and any additional intermediate members being integrally formed as a single unitary body with one another or with the two members or the two members and any additional intermediate members being attached to one another.

Certain terminology is used in the following description for convenience only and is not limiting. The words “right”, “left”, “lower”, and “upper” designate direction in the drawings to which reference is made. The words “inner” and “outer” refer to directions toward and away from, respectively, the geometric center of the described feature or device. The words “distal” and “proximal” refer to directions taken in context of the item described and, with regard to the instruments herein described, are typically based on the perspective of the practitioner using such instrument, with “proximal” indicating a position closer to the practitioner and “distal” indicating a position further from the practitioner. The terminology includes the above-listed words, derivatives thereof, and words of similar import.

Throughout the description and claims of this specification, the word “comprise” and variations of the word, such as “comprising” and “comprises”, means “including but not limited to”, and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The implementation was chosen and described in order to best explain the principles of the invention and the practical application, and to enable others of ordinary skill in the art to understand the invention for various implementations with various modifications as are suited to the particular use contemplated.

What is claimed is:

1. An introducer and flow redirection system for use in carotid angioplasty procedures, the system comprising:
a sheath assembly including;
a stabilizer,
a stabilizer foot coupled to the stabilizer and including a stabilizer eyelet configured to secure a suture,
a gripping mechanism coupled to the stabilizer remote from the stabilizer foot and including a valve eyelet configured to secure the suture,
an introducer sheath extending through the gripping mechanism, the stabilizer, and the stabilizer foot, and
an entry luer coupled to the introducer sheath;
wherein the gripping mechanism selectively locks the introducer sheath in place relative to the stabilizer;
a hemostatic valve including a hemostatic valve entry port coupled to the entry luer, a bypass port, and an access port;
a stopcock including a stopcock entry port, a collection port, and a stopcock valve providing selective communication between the stopcock entry port and the collection port; and
a collection bag connected to the collection port.

2. The introducer and flow redirection system of claim 1, wherein the stabilizer includes an elastomeric outer layer, a stainless steel coil, and a Polytetrafluoroethylene (PTFE) liner sized to receive the introducer sheath.

3. The introducer and flow redirection system of claim 1, wherein the stabilizer foot comprises silicone.

4. The introducer and flow redirection system of claim 1, wherein the introducer sheath includes printed indicators and a radiopaque marker.

5. The introducer and flow redirection system of claim 1, wherein the gripping mechanism is a Tuohy Borst valve.

6. The introducer and flow redirection system of claim 1, further comprising a flow control handle arranged between the collection port of the stopcock and the collection bag.

7. The introducer and flow redirection system of claim 6, wherein the flow control handle includes a flow control switch, a flow restrictor, and a flow visualization chamber.

8. The introducer and flow redirection system of claim 7, wherein the flow control switch defines a low flow position configured for use when an artery is unclamped and a high flow position configured for use when the artery is clamped.

9. The introducer and flow redirection system of claim 1, wherein the collection bag is non-aspirated.

10. The introducer and flow redirection system of claim 1, wherein the collection bag is configured to be positioned vertically below the introducer sheath to provide gravity flow from the collection port of the stopcock to the collection bag.

11. The introducer and flow redirection system of claim 1, further comprising an angioplasty device disposed within the introducer sheath.

12. The introducer and flow redirection system of claim 11, wherein the angioplasty device includes a self-collapsing filter and a self-expanding stent.

13. The introducer and flow redirection system of claim 12, wherein the self-collapsing filter comprises a filter membrane, the filter membrane having a pore size of between forty microns and one hundred microns.

14. The introducer and flow redirection system of claim 1, wherein the hemostatic valve is directly coupled to the entry luer, and the stopcock is directly coupled to the hemostatic valve.

15. The introducer and flow redirection system of claim 1, configured for introduction to a patient above a clavicle.

16. A method of performing a carotid angioplasty, comprising:
inserting an introducer sheath through a stabilizer including a stabilizer foot and a gripping mechanism;
inserting the introducer sheath into a first incision made in skin above a patient's clavicle and into a second incision made in a patient's carotid artery;
positioning the stabilizer foot adjacent the first incision and the second incision;
engaging the skin adjacent the first incision with a suture;
securing the suture to an eyelet of the stabilizer foot; and
providing a reverse flow from the introducer sheath to a collection bag via a hemostatic valve and a stopcock.

17. The method of claim 16, further comprising locking the introducer sheath in position relative to the stabilizer with the gripping mechanism.

18. The method of claim 16, further comprising adjusting a flow control switch arrangement between the collection bag and the stopcock from a low flow position to a high flow position during reverse flow.

19. The method of claim 16, further comprising introducing an angioplasty device including a self-collapsing filter and a self-expanding stent through the introducer sheath and expanding the self-collapsing filter at a location distal to a vascular lesion.

20. A carotid angioplasty kit, comprising:
a stabilizer including a stabilizer foot, a suture eyelet configured to secure a suture and maintain the stabilizer foot in position relative to an incision, and a gripping mechanism;
an introducer sheath sized to be engaged by the gripping mechanism and be received through the stabilizer;
a dilator sized to be received in the introducer sheath;
a hemostatic valve configured to be coupled to the introducer sheath;
a collection bag in fluid communication with the hemostatic valve; and
an angioplasty device sized to be disposed within the introducer sheath.

21. The carotid angioplasty kit of claim 20, further comprising a flow control handle configured to control reverse flow to the collection bag.
